(19) United States
(12) Patent Application Publication
(10) Pub. No.: US 2011/0277206 A1
(43) Pub. Date: Nov. 17, 2011

(54) GLOBAL POSITIONING SYSTEM GARMENT

(75) Inventor: SUSAN L. SOKOLOWSKI, Portland, OR (US)

(73) Assignee: NIKE, INC., Beaverton, OR (US)

(21) Appl. No.: 12/777,788

(22) Filed: May 11, 2010

(51) Int. Cl. A41D 1/00; A41D 27/20; A61B 5/04

(52) U.S. Cl. 2/69; 600/509; 2/250

(57) ABSTRACT

Global Positioning System (GPS) optimizing garments and methods for performing athletic training using a GPS signal optimizing garment are described. The garment may have a three-dimensional pocket positioned to promote a GPS receiver obtaining an optimal GPS signal. The pocket may also be positioned on the garment to prevent interference with the movement of the wearer of the garment. The garment may also include heart rate monitor tabs for retaining a heart rate monitor device.

### SUMMARY

Embodiments of the invention are defined by the claims below, not this summary. A high-level overview of various aspects of the invention are provided here for that reason, to provide an overview of the disclosure, and to introduce a selection of concepts that are further described below in the detailed-description section below. This summary is not intended to identify key features or essential features of the claimed subject matter, nor is it intended to be used as an aid in isolation to determine the scope of the claimed subject matter.

Global Positioning System (GPS) receivers use measurements from satellites to determine the position of the GPS receiver. GPS receivers typically determine their position by computing time delays between transmission and reception of signals transmitted from satellites and received by the receiver on or near the surface of the earth. The time delays are utilized to calculate the distance from the receiver to each of the satellites that are in view of the receiver. GPS receivers require a line of sight to the satellites in order to obtain a signal representative of the true distance from the satellite to the receiver. Therefore, any object in the path of the signal has the potential to interfere with the reception of that signal.

Handheld GPS receivers are often used during outdoor activities like camping, hiking and geocaching. In addition to route navigation, cyclists, runners, skiers and other athletes may utilize GPS devices during training or competitions to record training parameters like speed, distance and altitude. Despite their usefulness during sporting activities, it is often cumbersome to carry a GPS device during training and competition without interfering with athletic performance. Moreover, during high intensity sporting activities it is very difficult to hold a handheld GPS device in a position to receive a good satellite signal. As the use of GPS technology in conjunction with outdoor sporting activities increases, the need for a conveniently attaching a GPS receiver to an athlete while minimizing GPS signal blockage has arisen.

Embodiments of the present invention provide systems, and methods for transporting a GPS device utilizing a GPS signal optimizing garment. In various embodiments, the garment may have a three-dimensional pocket positioned to promote a GPS receiver obtaining an optimal GPS signal. In these embodiments, the pocket may be positioned on the garment to prevent interference with the movement of the wearer of the garment. In various embodiments, the garment may also include heart rate monitor tabs for retaining a heart rate monitor device.

### BRIEF DESCRIPTION OF THE SEVERAL VIEWS OF THE DRAWINGS

Illustrative embodiments of the present invention are described in detail below with reference to the attached drawing figures, which are incorporated by reference herein and wherein:

FIG. 1 depicts a front view of a garment embodying features of the present invention;

FIG. 2 depicts a rear view of a garment embodying features of the present invention;

FIG. 3 depicts a side view of a garment, in accordance with an embodiment of the present invention;

FIG. 4 depicts a rear view of another garment embodying features of the present invention;

FIG. 5 depicts an athlete training utilizing a GPS signal optimizing garment in accordance with an embodiment of the present invention;

FIG. 6 depicts a block diagram of an overall method of athletic training utilizing a GPS signal optimizing garment in accordance with an embodiment of the present invention; and

FIG. 7 depicts a front view of heart rate monitor tabs that may be used in a garment in accordance with the present invention.

### DETAILED DESCRIPTION

The subject matter of the present invention is described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies. Although the terms “step” and/or “block” or “module” etc. might be used herein to connote different components of methods or systems employed, the terms should not be interpreted as implying any particular order among or between various steps herein disclosed unless and except when the order of individual steps is explicitly described.

The present invention relates to a garment for carrying a GPS device during athletic training or competition to optimize the GPS signal without interfering with athletic activities. The invention further relates to methods for performing athletic training using a GPS signal optimizing garment. A garment in accordance with the present invention may have a three-dimensional pocket so as to hold the GPS receiver between the shoulders of an athlete training while wearing the garment positioned to promote a GPS receiver obtaining an optimal GPS signal.

Accordingly, in one aspect, the present invention provides a Global Positioning System (GPS) signal optimizing garment. The garment may be a shirt that extends around at least a portion of a wearer's torso in an as worn position, the shirt having a front and a back. The garment may provide a three-dimensional pocket attached to the back of the shirt at least one inch from a neckline of the back of the shirt, the pocket having a size to hold a GPS device.

In yet another aspect, the present invention provides a method for performing athletic training utilizing a GPS signal optimizing garment. The method includes providing a garment, the garment extending around at least a portion of a wearer's torso in an as worn position and having a front and a back. The garment further provides a three-dimensional pocket attached to the back of the garment at least one inch from a neckline of the shirt, the pocket having a size to hold a GPS device. The method also includes powering on a GPS device, inserting the GPS device in the pocket of the garment, and wearing the garment during athletic training and/or athletic competition.

GPS signal optimizing generally refers to enabling a GPS receiver to rapidly acquire the strongest GPS satellite signal, thereby increasing the accuracy of the receiver. GPS devices rely on the data received through the radio signals sent by a network of satellites orbiting the earth that send data regarding the altitude, longitude, and latitude of the GPS receiver. The satellite signals are sent with enough strength to reach the Earth's surface, however any object in the path of the signal has the potential to interfere with the reception of that signal. As such, factors like narrow streets and high buildings, wipers running back and forth across a windshield, tree canopies, mountains, and other large objects, even highway overpasses, can interfere with or interrupt the GPS signal. Similarly, the upper body of an athlete may interfere with GPS signal reception when the athlete carries a GPS device in his/her hand or in a pant or short pocket. Also, movement on an extremity can be a problem for tracking athletic performance. For example, GPS watches are generally considerably heavier than traditional watches and may significantly affect the training of high performance athletes. GPS watches are also inherently less accurate than GPS receivers attached to the core of the wearer's body for measuring parameters such as the distance travelled. Having to carry a GPS receiver in his/her hand can also interference with athletic training or competition. For example, during a high intensity activity such as running, holding a bulky handheld GPS device could significantly affect the speed and distance travelled of an athlete. Accordingly, the GPS signal reception of a receiver used by an athlete may be optimized by positioning the GPS receiver on the athlete's body so that an unobstructed line of sight from the receiver to the GPS satellites is provided. In addition, a GPS signal optimizing garment should prevent interference with the movement of the wearer of the garment.

Embodiments of the present invention provide GPS signal optimizing garments, and methods for performing athletic training using a GPS signal optimizing garment. Having briefly described an overview of embodiments of the present invention, an exemplary GPS signal optimizing garment is described below.

Referring to the drawings in general and FIGS. 1-5 in particular, an exemplary GPS signal optimizing garment is depicted in various views. While embodiments discussed herein refer to garments including a shirt, it will be understood that embodiments are not limited to any particular style or type of garment covering the upper body. For example, other embodiments may include jerseys, tech shirts, tank tops, unisuits, sport bras, jackets, vests and the like with GPS signal optimizing three-dimensional pockets. Further, the depictions in the drawings are for exemplary purposes only and are in no way meant to limit the scope of the present invention to any type of outdoor activity or athletic training technique. Further the materials to create the GPS devices, as well as the material properties of GPS devices are well known in the art and will not be discussed in further detail herein.

Referring now to FIG. 1, a front view of a garment embodying features of the present invention is illustrated and designated generally as reference numeral 100. Garment 100 includes a shirt 105, a three-dimensional pocket 110, with the shirt 105 including a front 115, back 117, sleeves 120, a neckline 125 and an opening 130. Garment 100 can be any type of material for use in athletic activity. For example, garment 100 may be constructed of form fitting material including but not limited to woven and non-woven knits, spandex, lycra, and athletic mesh. Shirt 105 extends around at least a portion of a wearers torso, with the front 115 covering the wearer's chest and back 117 covering the wearer's back. Shirt 105 can include sleeves 120 as shown; however shirt 105 can be long-sleeved or sleeveless. Shirt 105 may also include a neckline 125 that extends around a portion of a wearer's neck. Attached to the back 117 of shirt 105 may be a three-dimensional pocket 110. Pocket 110 may have a size to securely hold a GPS device. The back 117 of shirt 105 may include an opening 130 at the inner top edge of pocket 110. Opening 130 may allow insertion of a GPS device (not shown) into pocket 110. For optimal GPS signal reception by the GPS device, pocket 110 may be attached to the back 117 of shirt 105 one inch from neckline 125. In embodiments, pocket 110 may be located within three inches from the neckline 125 of shirt 105. Garment 100 may also include heart rate monitor tabs 135 for attaching a heart rate monitor device. Generally, heart rate monitor tabs may be shaped or sized for a particular type or brand of heart rate monitor device. In embodiments, heart rate monitor tabs 135 may be ‘universal’ tabs designed to facilitate receiving and retaining various types of known heart rate monitor device. In embodiments the heart rate monitor tabs 135 may be attached to the inside of garment 100 below sleeves 120. Heart rate monitor tabs 135 may enable a heart rate monitor device to be conveniently and comfortably attached to the chest of the wearer when 100 is in an as worn position. Garment 100 may include multiple heart rate monitor tabs 135 to ensure an optimal fit of a heart rate monitor device during athletic training and/or competition.

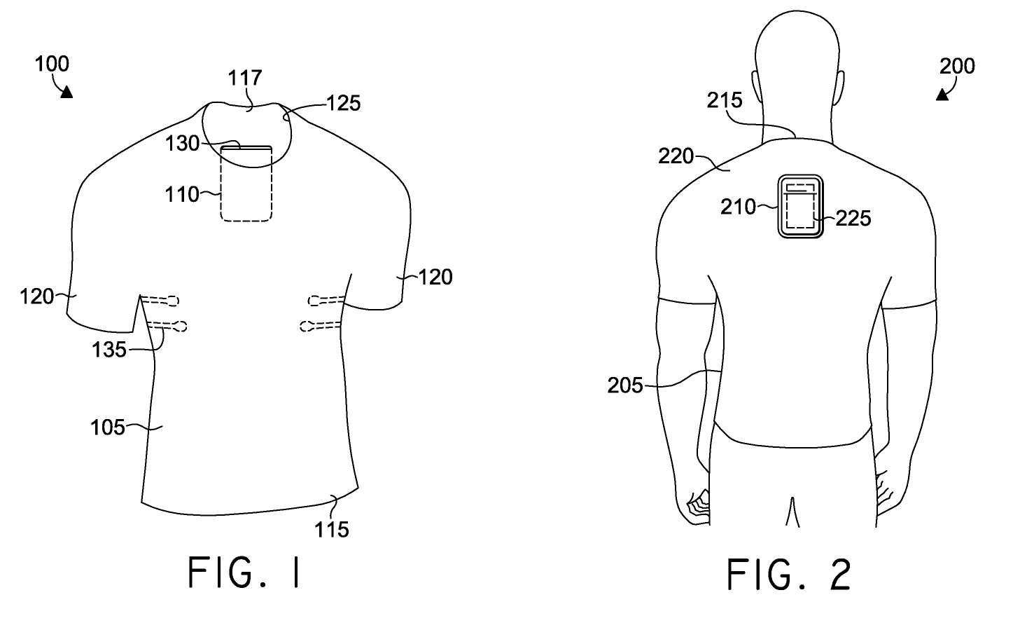

Turning now to FIG. 2, a rear view of a GPS signal optimizing garment embodying features of the present invention is illustrated and designated generally as reference number 200. Garment 200 includes a shirt 205, a three-dimensional pocket 210, a neckline 215, a back portion 220 of shirt 205 and a GPS device 225. Three-dimensional pocket 210 may be attached to the back 220 of shirt 205 between one half inch to three inches from the neckline 215. The placement of pocket 210 on garment 200 may increase the ability of GPS device 225 to receive signals. In embodiments the pocket 210 may be located between the shoulder blades of the back of a wearer in an as worn position. GPS device 225 may include GPS devices known in the art. In one example, pocket 210 may be constructed to hold a GPS device 225 with dimensions of at least 2 inches by 4 inches by 1 inch. In embodiments, GPS device 225 may include internal communication transmitters, receivers and processors.

Pocket 210 may be constructed of collapsible material and/or of yieldable fabric including but not limited to spacer mesh. Pocket 210 may be fabricated by cutting and sewing a patch of collapsible material into a three-dimensional shape and attaching the edges of the pocket 210 to the back of shirt 205. Pocket 210 may be placed on shirt 205 by stitching, gluing or any other technique.

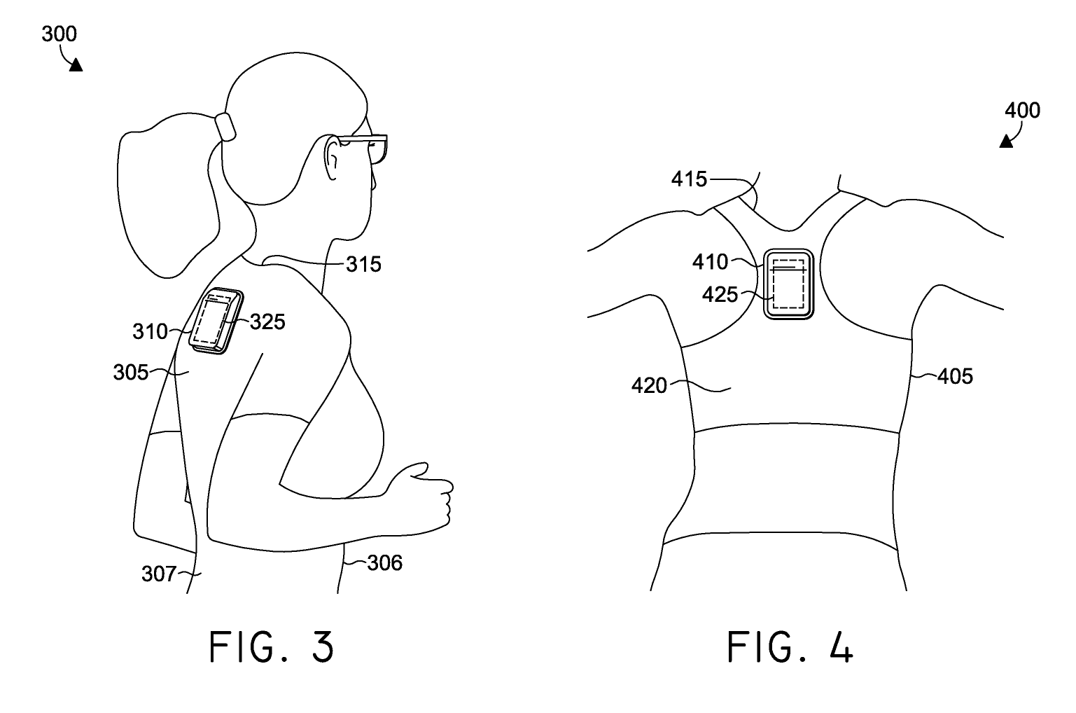

Turning now to FIG. 3, a side view of a GPS signal optimizing garment embodying features of the present invention is illustrated and designated generally as reference number 300. Garment 300 includes a shirt 305 that extends around a portion of a wearer's torso having a front 306, a back 307, a neckline 315. Attached to the back 307 of shirt 305 is a three-dimensional pocket 310. Pocket 310 may have a size to securely hold a GPS device. For optimal GPS signal reception by the GPS device, pocket 310 may be attached to the back 307 of shirt 305 at least one inch from neckline 315. The placement of pocket 310 may allow a wearer of garment 300 to transport a GPS device without having to hold the GPS device. Rather than uncomfortably carrying a GPS device in his/her hand, a wearer of garment 300 can transport a GPS device in pocket 310. As such, garment 300 may reduce GPS signal interference and limit interference with the athletic performance of the wearer.

Turning now to FIG. 4, a rear view of a another GPS signal optimizing garment embodying features of the present invention is illustrated and designated generally as reference number 400. Garment 400 includes a bra 405, a three-dimensional pocket 410, a neckline 415, a back portion 420 of bra 405 and a GPS device 425. Three-dimensional pocket 410 may be attached to the back 420 of bra 405 one inch from the neckline 415. The placement of pocket 410 on garment 400 may increase the ability of GPS device 425 to receive signals. In embodiments the pocket 410 may be located between the shoulder blades of the back of a wearer in an as worn position. GPS device 425 may include GPS devices known in the art. In embodiments, GPS device 425 may include internal communication transmitters, receivers and processors. In embodiments, GPS device 425 may also include an accelerometer for measuring acceleration forces. Data obtained from the accelerometer may be used in conjunction with the initial position and velocity from the GPS device to continuously determine the location and velocity of the device. This data may be use to provide location information of the GPS device that is independent of GPS data and may be particularly important in instances when the GPS device is unable to clearly receive GPS signals.

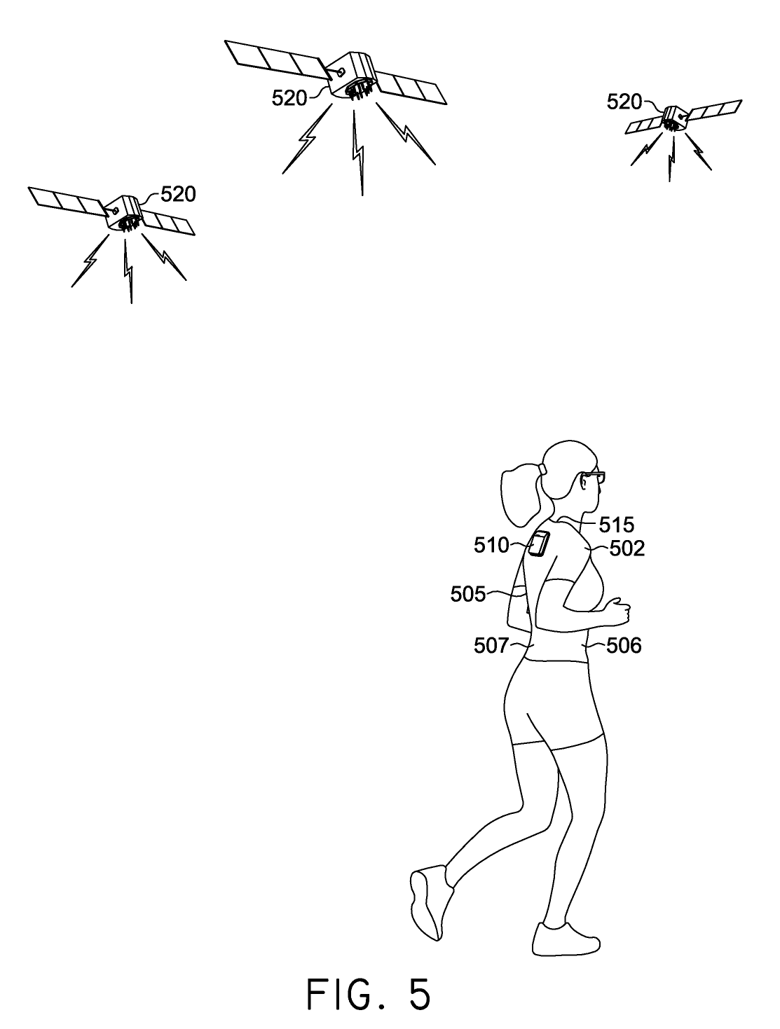

Turning now to FIG. 5, a diagram of an athlete training utilizing a GPS signal optimizing garment embodying features of the present invention is illustrated and designated generally as reference number 500. The training method includes a GPS signal optimizing garment 502 having a shirt 505 that extends around a portion of a wearer's torso. The shirt 505 may have a front 506, a back 507 and a neckline 515. Attached to the back 507 of shirt 505 is a three-dimensional pocket 510. Pocket 510 may have a size to securely hold a GPS device (not shown). The GPS device may receive radio signals from GPS satellites 520 and utilize the data received through the radio signals to determine parameters including the altitude, longitude, and/or latitude of the GPS receiver. Knowing these parameters may enable an athlete to determine their location, and record training parameters including but not limited to speed, altitude and distance. For optimal GPS signal reception by the GPS device, pocket 510 may be attached to the back 507 of shirt 305 at least one inch from neckline 515. The placement of pocket 510 may allow a wearer of garment 502 to train with a GPS device without having to hold the GPS device. Rather than uncomfortably carrying a GPS device in his/her hand that interferes with training or competition, an athlete wearing garment 502 can transport a GPS device in pocket 510. As such, garment 502 may reduce GPS signal interference and limit interference with the athletic performance of the wearer. The three-dimensional nature of pocket 510 also enables an athlete to securely wear a GPS device without experiencing the discomfort associated with having the GPS device in a two-dimensional pocket. While the diagram illustrates running, embodiments of the present invention may be used in other sporting activities where GPS signal optimization in conjunction with unencumbered hands would be useful. By way of example and not limitation, the present invention may be utilized in sporting activities including, skiing, rowing, and cycling.

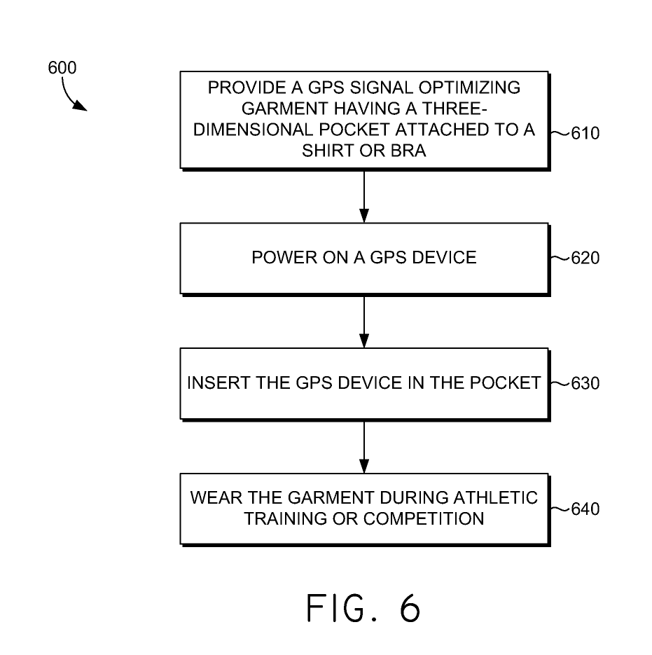

Referring now to FIG. 6, a block diagram is provided that illustrates a method 500 of athletic training utilizing a GPS signal optimizing garment in accordance with an embodiment of the present invention. Initially, as shown at block 510, a GPS signal optimizing garment may be provided. The garment may be a shirt or sport bra that extends around at least a portion of a wearer's torso having a front and a back. The garment may provide a three-dimensional pocket attached to the back of the shirt at least one inch from the neckline of the shirt, the pocket having a size to hold a GPS device. A GPS device may be powered on, as shown at block 620. The GPS device may be inserted in the pocket of the garment, as shown at block 630. The garment, containing the GPS device removably secured in the three-dimensional pocket, may be worn during athletic training or competition, as shown at block 640.

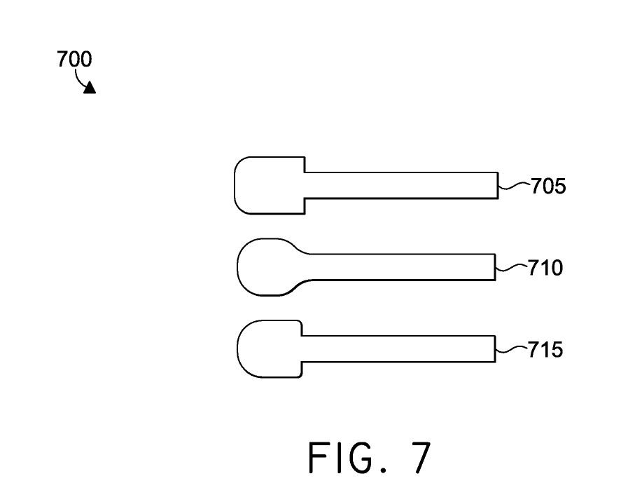

Turning now to FIG. 7, a front view of heart rate monitor tabs that may be used in a garment in accordance with the present invention are illustrated and designated generally as reference numeral 700. Heart rate monitor tabs 705 and 715 are illustrative examples of heart rate monitor tabs shaped or sized for a particular type or brand of heart rate monitor device. By way of example only, heart rate monitor tabs shaped like tab 705 may be used to receive and retain SUUNTO brand heart rate monitor chest straps, while heart rate monitor tabs shaped like tab 715 may be used for POLAR brand heart rate monitor chest straps. Heart rate monitor tab 710 is a further illustrative example of a universal heart rate monitor tab designed to facilitate retaining various types of known heart rate monitor devices. Heart rate monitor tabs 705, 710, and/or 715 may be affixed to the GPS optimizing garments of present invention.

Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

Embodiments of the present invention provide systems, and methods for transporting a GPS device utilizing a GPS signal optimizing garment. The invention further relates to methods for performing athletic training using a GPS signal optimizing garment. In various embodiments, the garment may have a three-dimensional pocket positioned to promote a GPS receiver obtaining an optimal GPS signal. In these embodiments, the pocket may be positioned on the garment to prevent interference with the movement of the wearer of the garment.

The invention claimed is:

1. A Global Positioning System (GPS) signal optimizing garment, the garment comprising:
a shirt that extends around at least a portion of a wearer's torso in an as worn position, the shirt having a front and a back; and
a three-dimensional pocket attached to the back of the shirt at least one inch from a neckline of the back of the shirt, the pocket having a size to hold a GPS device.

2. The garment of claim 1, wherein the pocket is located within three inches from the neckline of the back of the shirt.

3. The garment of claim 2, wherein the pocket is constructed of collapsible material, and wherein a patch of the collapsible material for the pocket is cut and sewn into a three-dimensional shape and attached at its edges to the shirt.

4. The garment of claim 1, wherein the garment is constructed of form fitting, athletic material.

5. The garment of claim 4, wherein the pocket is fabricated from yieldable fabric.

6. The garment of claim 5, wherein the pocket is fabricated from spacer mesh.

7. The garment of claim 4, further comprising one or more heart rate monitor tabs configured to retain a heart rate monitor device.

8. The garment of claim 7, wherein the one or more heart rate monitor tabs are attached to at least one side of an inner surface of the garment.

9. The garment of claim 6, wherein the pocket is constructed to hold a GPS device measuring at least 2 inches by 4 inches by 1 inch.

10. The garment of claim 6, wherein the pocket is located between the shoulder blades of the back of the wearer in an as worn position.

11. A system for transporting a Global Positioning System (GPS) device, the system comprising:
a GPS signal optimizing garment, the garment comprising:
a shirt that extends around at least a portion of a wearer's torso in an as worn position, the shirt having a front and a back;
a three-dimensional pocket attached to the back of the shirt between one half inch to three inches from the neckline of the shirt, the pocket having a size to hold a GPS device; and
the GPS device removably secured in the pocket.

12. The system of claim 11, wherein the GPS device includes an internal communication transmitter, and internal communication receiver and a GPS processor.

13. The system of claim 11, wherein the garment is constructed of form fitting, athletic material.

14. The system of claim 13, wherein the pocket is fabricated from yieldable fabric.

15. The system of claim 11, wherein the garment further comprises one or more heart rate monitor tabs configured to retain a heart rate monitor device.

16. The system of claim 11, wherein the pocket is constructed to hold a GPS device measuring at least 2 inches by 4 inches by 1 inch.

17. The system of claim 11, wherein the pocket is located between the shoulder blades of the back of the wearer in an as worn position.

18. A method for performing athletic training utilizing a Global Positioning System (GPS) signal optimizing garment, the method comprising:
providing a garment, the garment comprising:
a shirt that extends around at least a portion of a wearer's torso in an as worn position, the shirt having a front and a back; and
a three-dimensional pocket attached to the back of the shirt at least one inch from a neckline of the shirt, the pocket having a size to hold a GPS device;
powering on a GPS device;
inserting the GPS device in the pocket of the garment; and
wearing the garment during at least one of athletic training and athletic competition.

19. The method of claim 18, wherein the garment further comprises one or more heart rate monitor tabs configured to retain a heart rate monitor device.

20. The method of claim 18, wherein the pocket is fabricated from yieldable fabric and wherein the pocket is located between the shoulder blades of the back of the wearer in an as worn position.
